(19) United States
(12) Patent Application Publication
(10) Pub. No.: US 2023/0416838 A1
(43) Pub. Date: Dec. 28, 2023

(54) METHODS AND COMPOSITIONS FOR PREDICTING AND TREATING UVEAL MELANOMA

(71) Applicants: INSERM (INSTITUT NATIONAL DE LA SANTÉ ET DE LA RECHERCHE MÉDICALE), Paris (FR); UNIVERSITÉ COTE D'AZUR, Nice (FR)

(72) Inventors: Robert BALLOTTI, NICE (FR); Thomas STRUB, NICE (FR); Charlotte PANDIANI, NICE (FR); Corine BERTOLOTTO-BALLOTTI, NICE (FR)

(21) Appl. No.: 18/252,950

(22) PCT Filed: Nov. 15, 2021
(86) PCT No.: PCT/EP2021/081739

§ 371 Date: May 15, 2023

(30) Foreign Application Priority Data
Nov. 16, 2020 (EP) 20306384.7

(51) Int. Cl. C12Q 1/6886; C12Q 1/6841

(52) U.S. Cl. CPC C12Q 1/6886; C12Q 1/6841; C12Q 2600/158; C12Q 2600/118

(57) ABSTRACT

Here, in multi-scale analyses using single-cell RNA sequencing of six different primary uveal melanomas, inventors uncover a previously unrecognized intratumor heterogeneity at the genetic and transcriptomic level. They identify distinct transcriptional cell states and diverse tumor-associated populations in a subset of primary uveal melanomas.

### FIELD OF THE INVENTION

The invention is in the field of melanoma more particularly uveal melanoma. In particular, the invention provides methods and compositions for predicting and treating uveal melanoma.

### BACKGROUND OF THE INVENTION

Uveal melanoma is an aggressive and deadly neoplasm, which develops from melanocytes in the choroid. At diagnosis, only 1-3% of the patients have detectable metastases. However, despite successful treatment of the primary tumor, metastases, that display a pronounced liver tropism, develop in 50% of patients within a median time of 2.4 years¹. Once it has spread, there are no approved systemic treatments for uveal melanoma. Ninety percent of patients will die within 6 months after diagnosis of metastases (reviewed²⁻³). Therefore, rapid local treatments are crucial, as survival correlates with primary tumor size⁴. These observations imply that a subpopulation of uveal melanoma cells disseminates early during primary tumor progression. The characterization of these cells, the identification of specific markers and the discovery of targetable frailties are required to improve patient outcome. In skin melanomas, intra-tumoral heterogeneity has a profound impact on tumor evolution, development of metastases and drug resistance to therapy⁵⁻⁷. Thus, inter- and intra-tumoral heterogeneity, which is poorly characterized in uveal melanoma, may strongly contribute to the therapeutic impasse.

Therefore, the characterization of the different transcriptional states in primary melanomas may lead to the identification of new candidate biomarkers and reveal novel therapeutic targets.

### SUMMARY OF THE INVENTION

The invention relates to a method for predicting the survival time of a subject suffering from uveal melanoma and/or metastatic uveal melanoma comprising the steps of:
i) determining the score of SPP1, EMCN, SYNPR, CTC-340A15.2, HPGD, MTRNR2L8, PDE4DIP, COX6A2, AHCYL2, GSTA3 and H3F3A in a biological sample obtained from the subject;
ii) comparing the score quantified at step i) with its predetermined reference value and
iii) providing a good prognosis when the score of SPP1, EMCN, SYNPR, CTC-340A15.2, HPGD, MTRNR2L8, PDE4DIP, COX6A2, AHCYL2, GSTA3 and H3F3A is higher than its predetermined reference value or providing a bad prognosis when the score of SPP1, EMCN, SYNPR, CTC-340A15.2, HPGD, MTRNR2L8, PDE4DIP, COX6A2, AHCYL2, GSTA3 and H3F3A is lower than its predetermined reference value.

In particular, the invention is defined by claims.

### DETAILED DESCRIPTION OF THE INVENTION

Here, in multi-scale analyses using single-cell RNA sequencing of six different primary uveal melanomas, inventors uncover a previously unrecognized intratumor heterogeneity at the genetic and transcriptomic level. They identify distinct transcriptional cell states and diverse tumor-associated populations in a subset of primary uveal melanomas.

### Method for Predicting the Survival Time of a Subject Suffering from Uveal Melanoma and/or Metastatic Uveal Melanoma

Accordingly, in a first aspect, the invention relates to a method for predicting the survival time of a subject suffering from uveal melanoma and/or metastatic uveal melanoma comprising the steps of:
i) determining the score of SPP1, EMCN, SYNPR, CTC-340A15.2, HPGD, MTRNR2L8, PDE4DIP, COX6A2, AHCYL2, GSTA3 and H3F3 in a biological sample obtained from the subject;
ii) comparing the score quantified at step i) with its predetermined reference value and
iii) providing a good prognosis when the score of SPP1, EMCN, SYNPR, CTC-340A15.2, HPGD, MTRNR2L8, PDE4DIP, COX6A2, AHCYL2, GSTA3 and H3F3 is higher than its predetermined reference value or providing a bad prognosis when the score of SPP1, EMCN, SYNPR, CTC-340A15.2, HPGD, MTRNR2L8, PDE4DIP, COX6A2, AHCYL2, GSTA3 and H3F3 is lower than its predetermined reference value.

As used herein, the term “predicting” means that the subject to be analyzed by the method of the invention is allocated either into the group of subjects who will relapse, or into a group of subjects who will not relapse after a treatment.

The method is particularly suitable for predicting the duration of the overall survival (OS), progression-free survival (PFS) and/or the disease-free survival (DFS) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they have become cancer-free (achieved remission). DSF gives more specific information and is the number of people with a particular cancer who achieve remission. Also, progression-free survival (PFS) rates (the number of people who still have cancer, but their disease does not progress) include people who may have had some success with treatment, but the cancer has not disappeared completely.

As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

As used herein, the term “score” refers to a value allowing to determine the prognosis of a subject suffering from uveal melanoma and/or uveal melanoma resistant. Typically, to estimate the prognosis ability of PC1 genes, inventors use the top 10 up and down genes to estimate the survival for subjects in the TCGA cohort. Next, they plot a ROC curve and evaluated the Youden index (FIG. 2). The AUROC is 0.84 and the Youden index 0.63, thereby indicating that this PC1 score is of interest to estimate patients' prognosis. ROC curve is a graphic representation of the relation existing between the sensibility and the specificity of a test, that allows the determination and the comparison of the diagnostic performances of several tests. Different index associating sensitivity and specificity have been proposed. The most classic is that of Youden, which is 1 when the test is perfect. The exploration of this to the single cell analysis, cells with a PC1 score above the Youden index should be endowed with aggressive tumorigenic properties and convey a “poor prognosis”, while those with a PC1 score under the Youden index should be associated “good prognosis”.

In a particular embodiment, inventors used the principal component analysis (PCA) and examined the two first principal components, which they observed constituted the majority of the variance within the dataset. They identified SPP1, EMCN, SYNPR, CTC-340A15.2, HPGD, MTRNR2L8, PDE4DIP, COX6A2, AHCYL2, GSTA3 and H3F3 genes. Cellular function or disease analysis using Ingenuity® Pathway Analysis (IPA) software indicated that the signature with PCA was related to cell movement of tumor cell lines, migration of tumor cell lines, cell viability, cell survival, neoplasia of cells.

In a particular embodiment, the score and/or expression of SPP1, EMCN, SYNPR, CTC-340A15.2, HPGD, MTRNR2L8, PDE4DIP, COX6A2, AHCYL2, GSTA3 and H3F3A genes with a highest score is associated with long term survival (FIG. 1), whereas the expression of SPP1, EMCN, SYNPR, CTC-340A15.2, HPGD, MTRNR2L8, PDE4DIP, COX6A2, AHCYL2, GSTA3 and H3F3A genes with the lowest score is correlated with a short term survival. When the subject will have a long survival time, it is meant that the subject will have a “good prognosis”. In a further embodiment, the score is also called PC1 score.

As used herein, the term “SPP1” also known as Osteopontin refers to secreted phosphoprotein 1. SPP1 is involved in the interaction with multiple cell surface receptors that are ubiquitously expressed makes it an active player in many physiological and pathological processes including wound healing, bone turnover, tumorigenesis, inflammation, ischemia, and immune responses. The naturally occurring human SPP1 gene has a nucleotide sequence as shown in Genbank Accession numbers NM_001251830, NM 000582, NM_001040058, NM_001040060, NM_001251829. The naturally occurring human SPP1 protein has an aminoacid sequence as shown in Genbank Accession numbers NP_000573, NP_001035147, NP 001035149, NP_001238758, NP_001238759. The murine nucleotide and amino acid sequences have also been described (Genbank Accession numbers NM_001204201, NM_001204202, NM_001204203, NM_001204233, NM_009263 and NP_001191130, NP_001191131, NP_001191132, NP_001191162, NP_033289).

As used herein, the term “EMCN” refers to Endomucin. It is a mucin-like sialoglycoprotein that interferes with the assembly of focal adhesion complexes and inhibits interaction between cells and the extracellular matrix. NM_001159694, NM_016242 and the The naturally occurring human SPP1 protein has an aminoacid sequence as shown in Genbank Accession numbers NP_001153166 and NP_057326. The murine nucleotide and amino acid sequences have also been described (Genbank Accession numbers NM_001163522, NM_016885 and NP_001156994 NP_058581).

As used herein, the term “SYNPR” refers to Synaptoporin. Synaptoporin is a protein that in humans is encoded by the SYNPR gene. It is a channel protein of synaptic vesicles.

As used herein, the term “CTC-340A15.2” also known as LOC105377703. The gene is uncharacterized.

As used herein, the term “HPGD” refers to Hydroxyprostaglandin dehydrogenase 15-(NAD). HPGD is also known as HGNC ID, HGNC:5154, 15-hydroxyprostaglandin dehydrogenase [NAD+] is an enzyme that in humans is encoded by the HPGD gene. The naturally occurring human HPGD gene has a nucleotide sequence as shown in Genbank Accession numbers NM_000860, NM_001145816, NM_001256301, NM_001256305, NM_001256306. The naturally occurring human HPGD protein has an aminoacid sequence as shown in Genbank Accession numbers NP_000851, NP 001139288, NP_001243230, NP 001243234. NP 001243235.

As used herein, the term “MTRNR2L8” is a protein in humans that is encoded by the MTRNR2L8 gene. MTRNR2L8 plays a role as a neuroprotective and antiapoptotic factor.

As used herein, the term “PDE4DIP” also known as myomegalin, phosphodiesterase 4D-interacting protein or cardiomyopathy-associated protein 2 is a protein that in humans is encoded by the PDE4DIP gene. PDE4DIP plays a role in the formation of microtubules from the centrosome. The naturally occurring human PDE4DIP gene has a nucleotide sequence as shown in Genbank Accession numbers NM_001002810, NM_001002811, NM_001002812, NM_001195260, NM_001195261. The naturally occurring human PDE4DIP protein has an aminoacid sequence as shown in Genbank Accession numbers NP_001002810, NP_001002811, NP_001002812, NP_001182189, NP_001182190. The murine nucleotide and amino acid sequences have also been described (Genbank Accession numbers NM_001039376, NM_001110163, NM_001289701, NM_001289702, NM_031401 and NP_001034465, NP_001276630, NP_001276631, NP_835181).

As used herein, the term “COX6A2” refers to Cytochrome c oxidase subunit VIa polypeptide 2 i. Cytochrome c oxidase (COX) is the terminal enzyme of the mitochondrial respiratory chain. The naturally occurring human COX6A2 gene has a nucleotide sequence as shown in Genbank Accession number NM_005205 and the naturally occurring human COX6A2 protein has an aminoacid sequence as shown in Genbank Accession number NM_009943. The murine nucleotide and amino acid sequences have also been described (Genbank Accession numbers NM_009943 and NP_034073).

As used herein, the term “AHCYL2” refers to S-Adenosylhomocysteine Hydrolase-Like 2. It regulates the electrogenic sodium/bicarbonate cotransporter SLC4A4 activity and Mg2+-sensitivity.

As used herein, the term “H3F3A” refers to histone H3.3. It plays an essential role in maintaining genome integrity during mammalian development.

As used herein, the term “GSTA3” refers to glutathione S-transferase A3 which is an enzyme that in humans is encoded by the GSTA3 gene. Cytosolic and membrane-bound forms of glutathione S-transferase are encoded by two distinct supergene families. These enzymes are involved in cellular defense against toxic, carcinogenic, and pharmacologically active electrophilic compounds. At present, eight distinct classes of the soluble cytoplasmic mammalian glutathione S-transferases have been identified: alpha, kappa, mu, omega, pi, sigma, theta and zeta. The naturally occurring human GSTA3 gene has a nucleotide sequence as shown in Genbank Accession number NM_000847 and the naturally occurring human GSTA3 protein has an aminoacid sequence as shown in Genbank Accession number NP_000838.

Accordingly, in a particular embodiment, the invention relates to a method for predicting the survival time of a subject suffering from uveal melanoma and/or metastatic uveal melanoma comprising the steps of:
i) determining the score of SPP1, EMCN, SYNPR, CTC-340A15.2, HPGD, MTRNR2L8, PDE4DIP, COX6A2, AHCYL2, GSTA3 and H3F3A in a biological sample obtained from the subject;
ii) comparing the score quantified at step i) with its predetermined reference value and
iii) providing a good prognosis when the score is highest compared to its predetermined reference value.

As used herein, the term “subject” denotes a mammal, such as a rodent, a feline, a canine, and a primate. Particularly, the subject according to the invention is a human. More particularly, the subject according to the invention has or is susceptible to have uveal melanoma.

In particular embodiment, the subject has or is susceptible to have uveal melanoma resistant.

In a particular embodiment, the subject has or is susceptible to have metastatic melanoma.

As used herein, the term “uveal melanoma” refers to a disease in which malignant (cancer) cells form in the tissues of the eye. It is an aggressive and deadly neoplasm, which develops from melanocytes in the choroid. At diagnosis, only 1-3% of the patients have detectable metastases.

As used herein, the term “metastasis” refers to the spread of cancer cells from a primary site and the formation of new tumors in another region of the body. Metastasis is responsible for as much as 90% of cancer-associated mortality. The liver is often the first metastatic site in patients with uveal melanoma. Accordingly, metastatic uveal melanoma refers migration of ciliary or choroid cells to the liver and induces liver metastasis.

In a particular embodiment, the uveal melanoma is resistant. As used herein, the term “uveal melanoma resistant” refers to uveal melanoma which does not respond to a treatment. The cancer may be resistant at the beginning of treatment or it may become resistant during treatment. The resistance to drug leads to rapid progression of metastatic of uveal melanoma.

The resistance of cancer for the medication is caused by mutations in the gene which are involved in the proliferation, divisions or differentiation of cells.

In a particular embodiment, the uvea melanoma resistant has at least one mutation in the five following genes: BAP1, EIF1AX, GNA11, GNAQ, and/or SF3B1.

In another embodiment, the uveal melanoma is resistant to a treatment with an immune check point inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

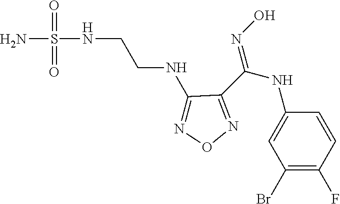 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

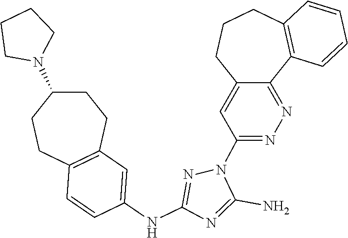 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “expression level” refers to the expression level of SPP1, EMCN, SYNPR, CTC-340A15.2, HPGD, MTRNR2L8, PDE4DIP, COX6A2, AHCYL2, GSTA3, H3F3A (PC1 signature having highest score). Typically, the expression level of the SPP1, EMCN, SYNPR, CTC-340A15.2, HPGD, MTRNR2L8, PDE4DIP, COX6A2, AHCYL2, H3F3A, GSTA3 gene may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

As used herein, the term “biological sample” refers to any sample obtained from a subject, such as a serum sample, a plasma sample, a urine sample, a blood sample, a lymph sample, or a tissue biopsy. In a particular embodiment, biological sample for the determination of an expression level include samples such as a blood sample, a lymph sample, or a tumor biopsy sample.

In a particular embodiment, the biological sample is a blood sample, more particularly, peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

In a particular embodiment, the biological sample is a tumor biopsy sample.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of
a) providing a collection of tumor tissue samples from subject suffering from melanoma;
b) providing, for each tumor tissue sample provided at step a), information relating to the actual clinical outcome for the corresponding subject (i.e. the duration of the disease-free survival (DFS) and/or the overall survival (OS));
c) providing a serial of arbitrary quantification values;
d) quantifying the cell density for each tumor tissue sample contained in the collection provided at step a);
e) classifying said tumor tissue samples in two groups for one specific arbitrary quantification value provided at step c), respectively: (i) a first group comprising tumor tissue samples that exhibit a quantification value for level that is lower than the said arbitrary quantification value contained in the said serial of quantification values; (ii) a second group comprising tumor tissue samples that exhibit a quantification value for said level that is higher than the said arbitrary quantification value contained in the said serial of quantification values; whereby two groups of tumor tissue samples are obtained for the said specific quantification value, wherein the tumor tissue samples of each group are separately enumerated;
f) calculating the statistical significance between (i) the quantification value obtained at step e) and (ii) the actual clinical outcome of the subjects from which tumor tissue samples contained in the first and second groups defined at step f) derive;
g) reiterating steps f) and g) until every arbitrary quantification value provided at step d) is tested;
h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 tumor tissue samples of 100 subjects. The 100 samples are ranked according to the cell density. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

In a particular embodiment, the expression level of SPP1, EMCN, SYNPR, CTC-340A15.2, HPGD, MTRNR2L8, PDE4DIP, COX6A2, AHCYL2, H3F3A, GSTA3 is performed by RNA scope assays.

In a particular embodiment, the method according to the invention further comprises a step of classification of subject by an algorithm and determining whether a subject will have a long survival time.

Typically, the method of the present invention comprises a) quantifying the expression level of the SPP1, EMCN, SYNPR, CTC-340A15.2, HPGD, MTRNR2L8, PDE4DIP, COX6A2, AHCYL2, H3F3A, GSTA3 in the biological sample; b) implementing a classification algorithm on data comprising the quantified of SPP1, EMCN, SYNPR, CTC-340A15.2, HPGD, MTRNR2L8, PDE4DIP, COX6A2, AHCYL2, H3F3A, GSTA3 levels so as to obtain an algorithm output; c) determining the probability that the subject have a long survival time from the algorithm output of step b).

In some embodiments, the method according to the invention wherein the algorithm is selected from Linear Discriminant Analysis (LDA), Topological Data Analysis (TDA), Neural Networks, Support Vector Machine (SVM) algorithm and Random Forests algorithm (RF). selected from Linear Discriminant Analysis (LDA), Topological Data Analysis (TDA), Neural Networks, Support Vector Machine (SVM) algorithm and Random Forests algorithm (RF).

In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

The algorithm can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

### Methods for Treating Uveal Melanoma and/or Metastatic Uveal Melanoma

In a second aspect, the invention relates to a method for treating uveal melanoma and/or metastatic uveal melanoma in a subject in need thereof comprising a step of administering said subject with a therapeutically effective amount of an activator of SPP1, EMCN, SYNPR, CTC-340A15.2, HPGD, MTRNR2L8, PDE4DIP, COX6A2, AHCYL2, GSTA3 and H3F3.

In a particular embodiment, the subject is identified as having a short survival time (thus bad prognosis) by performing the method as described above.

Typically, the invention relates to a method for treating uveal melanoma and/or metastatic uveal melanoma in a subject in need thereof comprising the following steps:
i) determining the score of SPP1, EMCN, SYNPR, CTC-340A15.2, HPGD, MTRNR2L8, PDE4DIP, COX6A2, AHCYL2, GSTA3 and H3F3 in a biological sample obtained from the subject;
ii) comparing the score quantified at step i) with its predetermined reference value;
iii) providing a conclusion on prognosis when the score of SPP1, EMCN, SYNPR, CTC-340A15.2, HPGD, MTRNR2L8, PDE4DIP, COX6A2, AHCYL2, GSTA3 and H3F3 is lowest than their predetermined reference value; and
iv)) administering to said subject a therapeutically effective amount of an activator of SPP1, EMCN, SYNPR, CTC-340A15.2, HPGD, MTRNR2L8, PDE4DIP, COX6A2, AHCYL2, GSTA3 and/or H3F3.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The term “activator of SPP1, EMCN, SYNPR, CTC-340A15.2, HPGD, MTRNR2L8, PDE4DIP, COX6A2, AHCYL2, H3F3A, GSTA3” refers to a natural or synthetic compound that has a biological effect to stimulate/activate the activity or the expression of SPP1, EMCN, SYNPR, CTC-340A15.2, HPGD, MTRNR2L8, PDE4DIP, COX6A2, AHCYL2, GSTA3, H3F3A.

In a particular embodiment, the activator of SPP1, EMCN, SYNPR, CTC-340A15.2, HPGD, MTRNR2L8, PDE4DIP, COX6A2, AHCYL2, H3F3A, GSTA3 signature (having a lowest PC1 score) is selected from the group consisting but not limited to: a small organic molecule, an aptamer an antibody, a peptide or a polypeptide.

As used herein, the term “subject” denotes a mammal, such as a rodent, a feline, a canine, and a primate. Particularly, the subject according to the invention is a human. More particularly, the subject according to the invention has or susceptible to have uveal melanoma.

In a particular embodiment, the subject has or susceptible to have metastatic uveal melanoma.

In a particular embodiment, the subject has or susceptible to have uveal melanoma resistant to at least one of the treatments as described above.

As used herein the terms “administering” or “administration” refer to the act of injecting or otherwise physically delivering a substance as it exists outside the body (e.g., activator of SPP1, EMCN, SYNPR, CTC-340A15.2, HPGD, MTRNR2L8, PDE4DIP, COX6A2, AHCYL2, GSTA3 and/or H3F3A) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of an activator of of SPP1, EMCN, SYNPR, CTC-340A15.2, HPGD, MTRNR2L8, PDE4DIP, COX6A2, AHCYL2, H3F3A, GSTA3 for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Combined Preparation

In a third aspect, the invention relates to i) an activator of SPP1, EMCN, SYNPR, CTC-340A15.2, HPGD, MTRNR2L8, PDE4DIP, COX6A2, AHCYL2, H3F3A, GSTA3 and ii) a classical treatment as a combined preparation for use in the treatment of uveal melanoma and/or metastatic uveal melanoma.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 according to the invention, and ii) a classical treatment as a combined preparation for simultaneous, separate or sequential use in the treatment of uveal melanoma and/or metastatic uveal melanoma.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 for use according to the invention, and ii) a classical treatment as a combined preparation for simultaneous, separate or sequential use in the treatment of uveal melanoma resistant.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The activator of an activator of SPP1, EMCN, SYNPR, CTC-340A15.2, HPGD, MTRNR2L8, PDE4DIP, COX6A2, AHCYL2, H3F3A, GSTA3 as described above can be used alone as a single activator or in combination with other a classical treatment. When several activators are used, a mixture of activators is obtained. In the case of multi-therapy (for example, bi-, tri- or quadritherapy), at least one other activator can accompany the SPP1, EMCN, SYNPR, CTC-340A15.2, HPGD, MTRNR2L8, PDE4DIP, COX6A2, AHCYL2, H3F3A, GSTA3 activator.

As used herein, the term “classical treatment” refers to treatments well known in the art and used to treat melanoma. In the context of the invention, the classical treatment refers to radiation therapy, immunotherapy or chemotherapy.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) immunotherapy as a combined preparation for use in the treatment of uveal melanoma, metastatic uveal melanoma and/or uveal melanoma resistant.

In a particular embodiment, the combined preparation for use in the treatment of uveal melanoma, metastatic uveal melanoma and/or uveal melanoma resistant, wherein, said modulator is an activator of SPP1, EMCN, SYNPR, CTC-340A15.2, HPGD, MTRNR2L8, PDE4DIP, COX6A2, AHCYL2, H3F3A, GSTA3 activity and/or expression.

In a particular embodiment, the combined preparation for use in the treatment of uveal melanoma, metastatic uveal melanoma and/or uveal melanoma resistant, wherein, said modulator is an activator of SPP1, EMCN, SYNPR, CTC-340A15.2, HPGD, MTRNR2L8, PDE4DIP, COX6A2, AHCYL2, H3F3A, GSTA3.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) immunotherapy as a combined preparation for use in the treatment of uveal melanoma, metastatic uveal melanoma and/or uveal melanoma resistant.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) immunotherapy as a combined preparation according to the invention for simultaneous, separate or sequential use in the treatment of uveal melanoma, metastatic uveal melanoma and/or uveal melanoma resistant.

As used herein, the term “immunotherapy” has its general meaning in the art and refers to the treatment that consists in administering an immunogenic agent i.e. an agent capable of inducing, enhancing, suppressing or otherwise modifying an immune response. In a particular embodiment, the immunotherapy consists of use of an immune check point inhibitor as described above.

In a particular embodiment, the invention relates i) an activator of SPP1, EMCN, SYNPR, CTC-340A15.2, HPGD, MTRNR2L8, PDE4DIP, COX6A2, AHCYL2, H3F3A, GSTA3 and ii) a chemotherapy used as a combined preparation for use in the treatment of uveal melanoma, metastatic uveal melanoma and/or uveal melanoma resistant.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) chemotherapy as a combined preparation according to the invention for simultaneous, separate or sequential use in the use in the treatment of uveal melanoma, metastatic uveal melanoma and/or uveal melanoma resistant.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the invention relates i) an activator of SPP1, EMCN, SYNPR, CTC-340A15.2, HPGD, MTRNR2L8, PDE4DIP, COX6A2, AHCYL2, H3F3A, GSTA3 and ii) a radiotherapy used as a combined preparation for use in the treatment of uveal melanoma, metastatic uveal melanoma and/or uveal melanoma resistant.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) radiotherapy as a combined preparation according to the invention for simultaneous, separate or sequential use in the use in the treatment of uveal melanoma, metastatic uveal melanoma and/or uveal melanoma resistant.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates i) an activator of SPP1, EMCN, SYNPR, CTC-340A15.2, HPGD, MTRNR2L8, PDE4DIP, COX6A2, AHCYL2, H3F3A, GSTA3 and ii) an immune checkpoint inhibitor used as a combined preparation for the treatment of uveal melanoma, metastatic uveal melanoma and/or uveal melanoma resistant.

In a particular embodiment, the immune checkpoint inhibitor as a combined preparation according to the invention, wherein the immune checkpoint inhibitor is selected from the group consisting of but not limited to: Nivolumab (Opdivo®, BMS), Pembrolizumab (also called Lambrolizumab, KEYTRUDA® or MK-3475, MERCK). Atezolizumab (MPDL3280A, Genentech/Roche), Durvalumab (AZD9291, AstraZeneca), Avelumab (also known as MSB0010718C, Merck) and BMS-936559 (BMS). In particular embodiment, immune checkpoint inhibitor is described above.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) a MEK inhibitor as a combined preparation for use in the treatment of uveal melanoma, metastatic uveal melanoma and/or uveal melanoma resistant.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) a MEK inhibitor as a combined preparation according to the invention for simultaneous, separate or sequential use in the treatment of uveal melanoma, metastatic uveal melanoma and/or uveal melanoma resistant.

As used herein, the term “MEK” refers to Mitogen-activated protein kinase, also known as MAP2K, MEK, MAPKK. It is a kinase enzyme which phosphorylates mitogenactivated protein kinase (MAPK). The inhibitors of MEK are well known in the art. In a particular embodiment, the melanoma is resistant to a treatment with trametinib also known as mekinist which is commercialized by GSK. In a particular embodiment, the melanoma is resistant to a treatment with cobimetinib also known as cotellic commercialized by Genentech. In a particular embodiment, the melanoma is resistant to a treatment with Binimetinib also knowns as MEK162, ARRY-162 is developed by Array Biopharma.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) an HDAC inhibitor as a combined preparation for use in the treatment of uveal melanoma, metastatic uveal melanoma and/or uveal melanoma resistant.

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) an HDAC inhibitor as a combined preparation according to the invention for simultaneous, separate or sequential use in the treatment of uveal melanoma, metastatic uveal melanoma and/or uveal melanoma resistant.

As used herein, the term “HDAC” refers to histone deacetylases which is a class of enzymes that remove acetyl groups (O═C—CH3) from an ε-N-acetyl lysine amino acid on a histone, allowing the histones to wrap the DNA more tightly.

As used herein, the term histone “histone deacetylase inhibitor” called also HDACi, refers to a class of compounds that interfere with the function of histone deacetylase. Histone deacetylases (HDACs) play important roles in transcriptional regulation and pathogenesis of cancer. Typically, inhibitors of HDACs modulate transcription and induce cell growth arrest, differentiation and apoptosis. HDACis also enhance the cytotoxic effects of therapeutic agents used in cancer treatment, including radiation and chemotherapeutic drugs. In a particular embodiment, the histone deacetylase inhibitor is valproic acid (VPA). The term “valproic acid” refers to acid-2-propylpentanoic (C₈H₁₆O₂), 5 which has the following CAS number and formula 99-66-1 in the art:

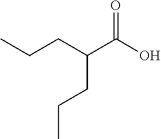

In a particular embodiment, the HDAC inhibitor is suberoylanilide hydroxamic acid, also called Vorinostat (N-Hydroxy-N′-phenyloctanediamide) was the first histone deacetylase inhibitor approved by the U.S. Food and Drug Administration (FDA) on 2006 (Marchion D C et al 2004; Valente et al 2014).

In a particular embodiment the HDAC inhibitor is Panobinostat (LBH-589) has received the FDA approval on 2015 and has the structure as described in Valente et al 2014.

In a particular embodiment the HDAC inhibitor is Givinostat (ITF2357) has been granted as an orphan drug in the European Union (Leoni et al 2005; Valente et al 2014).

In a particular embodiment the HDAC inhibitor is Belinostat also called Beleodaq (PXD-101) has received the FDA approval on 2014 (Ja et al 2003; Valente et al 2014).

In a particular embodiment the HDAC inhibitor is Entinostat (as SNDX-275 or MS-275). This molecule has the following chemical formula (C₂₁H₂₀N₄O₃) and has structure as described in Valente et al 2014.

In a particular embodiment the HDAC inhibitor is Mocetinostat (MGCD01030) having the following chemical formula (C₂₃H₂₀N₆O) (Valente et al 2014).

In a particular embodiment the HDAC inhibitor is Practinostat (SB939) having the following chemical formula (C₂₀H₃₀N₄O₂) and the structure as described in Diermayr et al 2012.

In a particular embodiment the HDAC inhibitor is Chidamide (CS055/HBI-8000) having the following chemical formula (C₂₂H₁₉FN₄O₂).

In a particular embodiment the HDAC inhibitor is Quisinostat (JNJ-26481585) having the following chemical formula (C₂₁H₂₆N₆O₂).

In a particular embodiment the HDAC inhibitor is Abexinostat (PCI24781) having the following chemical formula (C₂₁H₂₃N₃O₅) (Valente et al 2014).

In a particular embodiment the HDAC inhibitor is CHR-3996 having the following chemical formula (C₂₀H₁₉FN₆O₂) (Moffat D et al 2010; Banerji et al 2012).

In a particular embodiment the HDAC inhibitor is AR-42 having the following chemical formula (C₁₈H₂₀N₂O₃) (Lin et al 2012).

In a particular embodiment, i) an activator of SPP1, EMCN, SYNPR, CTC-340A15.2, HPGD, MTRNR2L8, PDE4DIP, COX6A2, AHCYL2, H3F3A, GSTA3 and ii) a calcium channel blocker, as a combined preparation for use in the treatment of uveal melanoma, metastatic uveal melanoma and/or uveal melanoma resistant.

As used herein, the term “calcium channel blocker” (CCB) refers to calcium channel antagonists or calcium antagonists that disrupt the movement of calcium (Ca2+) through calcium channels.

In a particular embodiment, the calcium channel blocker is selected from the following group consisting of but not limited to Amlodipine (Norvasc), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Efonidipine (Landel), Felodipine (Plendil), Isradipine (DynaCirc, Prescal), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas), Fendiline, Gallopamil, Verapamil (Calan, Isoptin) or Diltiazem.

### Pharmaceutical Composition

The activator of SPP1, EMCN, SYNPR, CTC-340A15.2, HPGD, MTRNR2L8, PDE4DIP, COX6A2, AHCYL2, H3F3A, GSTA3 for use according to the invention alone and/or combined with classical treatment as described above may be combined with pharmaceutically acceptable excipients, and optionally sustained-release matrices, such as biodegradable polymers, to form pharmaceutical compositions.

Accordingly, in a fourth aspect, the invention relates to a pharmaceutical composition comprising an activator of SPP1, EMCN, SYNPR, CTC-340A15.2, HPGD, MTRNR2L8, PDE4DIP, COX6A2, AHCYL2, H3F3A, GSTA3 for use in the treatment of uveal melanoma and/or metastatic uveal melanoma.

In a particular embodiment, the pharmaceutical composition according the invention, wherein the activator is an activator of SPP1, EMCN, SYNPR, CTC-340A15.2, HPGD, MTRNR2L8, PDE4DIP, COX6A2, AHCYL2, H3F3A, GSTA3 activity and/or expression.

In a particular embodiment, the pharmaceutical composition according the invention comprising i) an activator is an activator of SPP1, EMCN, SYNPR, CTC-340A15.2, HPGD, MTRNR2L8, PDE4DIP, COX6A2, AHCYL2, H3F3A, GSTA3 and ii) a classical treatment, as a combined preparation.

The activator of SPP1, EMCN, SYNPR, CTC-340A15.2, HPGD, MTRNR2L8, PDE4DIP, COX6A2, AHCYL2, H3F3A, GSTA3 and the combined preparation as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Kit for Predicting and Treating Uveal Melanoma

In a fifth aspect, the invention relates to a kit suitable to predict the survival time of a subject suffering or susceptible to suffer from uveal melanoma and/or metastatic uveal melanoma.

Accordingly, the invention relates to a kit for use in the method for predicting the survival time of a subject having or susceptible to have uveal melanoma and/or metastatic uveal melanoma said kit comprising a reagent that specifically reacts with SPP1, EMCN, SYNPR, CTC-340A15.2, HPGD, MTRNR2L8, PDE4DIP, COX6A2, AHCYL2, GSTA3, H3F3A, mRNA or protein and instructions to perform the predicting method of the survival time according to the method as described above.

The kit for the use according to the invention, wherein the reagent that specifically reacts with SPP1, EMCN, SYNPR, CTC-340A15.2, HPGD, MTRNR2L8, PDE4DIP, COX6A2, AHCYL2, H3F3A, GSTA3 and/or B2M mRNA or protein is selected from the group consisting of oligonucleotide probes that specifically hybridize to SPP1, EMCN, SYNPR, CTC-340A15.2, HPGD, MTRNR2L8, PDE4DIP, COX6A2, AHCYL2, GSTA3, H3F3A mRNA transcripts, oligonucleotide primers that specifically amplify SPP1, EMCN, SYNPR, CTC-340A15.2, HPGD, MTRNR2L8, PDE4DIP, COX6A2, AHCYL2, GSTA3, H3F3A mRNA transcripts, antibodies that specifically recognize/bind the SPP1, EMCN, SYNPR, CTC-340A15.2, HPGD, MTRNR2L8, PDE4DIP, COX6A2, AHCYL2, GSTA3, H3F3A, protein, and SPP1, EMCN, SYNPR, CTC-340A15.2, HPGD, MTRNR2L8, PDE4DIP, COX6A2, AHCYL2, GSTA3, H3F3A, -binding peptides that specifically bind to the SPP1, EMCN, SYNPR, CTC-340A15.2, HPGD, MTRNR2L8, PDE4DIP, COX6A2, AHCYL2, GSTA3, H3F3A, protein.

### Method of Screening

In a another aspect, the present invention relates to a method of screening a drug suitable for the treatment of melanoma, aggressive/invasive melanoma, metastatic melanoma or melanoma resistant comprising i) providing a test compound and ii) determining the ability of said test compound to activate the expression and/or activity of SPP1, EMCN, SYNPR, CTC-340A15.2, HPGD, MTRNR2L8, PDE4DIP, COX6A2, AHCYL2, GSTA3, H3F3A.

Typically, such test compound is able to activate the expression and/or activity of SPP1, EMCN, SYNPR, CTC-340A15.2, HPGD, MTRNR2L8, PDE4DIP, COX6A2, AHCYL2, GSTA3, H3F3A.

Any biological assay well known in the art could be suitable for determining the ability of the test compound to activate SPP1, EMCN, SYNPR, CTC-340A15.2, HPGD, MTRNR2L8, PDE4DIP, COX6A2, AHCYL2, GSTA3, H3F3A. In some embodiments, the assay first comprises determining the ability of the test compound to bind to SPP1, EMCN, SYNPR, CTC-340A15.2, HPGD, MTRNR2L8, PDE4DIP, COX6A2, AHCYL2, GSTA3, H3F3A. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of activating the activity of SPP1, EMCN, SYNPR, CTC-340A15.2, HPGD, MTRNR2L8, PDE4DIP, COX6A2, AHCYL2, GSTA3, H3F3A, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Single-cell RNA-seq uncovers poor prognosis cell subpopulations. (A) Kaplan Meier survival plot of candidate genes with high PC1 scores (lower left quadrant). (B) Kaplan Meier survival plot of candidate genes with low PC1 scores (upper right quadrant) in PC1.

FIG. 2: Death predictive value of the PC1 score in TCGA uveal melanoma cohort using the ROC curve. Receiver Operating Characteristic (ROC) curve, using the PC1 score and the death status illustrates the prediction of patient's death by uveal melanoma in the TCGA cohort. The PC1 score was based on the top 10 up and down genes. AUC, Area Under the Curve.

FIG. 3: Single-cell RNA-seq uncovers poor prognosis cell subpopulations. (A-C) Ingenuity pathway analysis (IPA) on the PC1 genes (z-score −1/+1; 268 genes up; 15 down). The significantly involved disease and cellular functions are shown with the z-score heatmap that ranges from dark blue (low expression) to dark orange (high expression).

FIG. 4: Cluster characterisation. Related to FIG. 1. (A-B). Kaplan-Meier survival plot for the top 25 genes of the indicated clusters.

### EXAMPLE

Material and Methods

Sample Collection and Processing

Tumor tissues were obtained from patients diagnosed with ocular melanoma, after written informed consent was obtained from the Nice CHU hospital. Surgically removed primary tumor was kept in sterile and RPMI medium. To isolate viable single cells that are suitable for high-quality single-cell RNA-seq (scRNA-seq), tumor tissues were processed immediately after surgical resection to prepare individual cells suspension. Briefly, samples were minced on a plate and enzymatically digested with collagenase A (Roche), dispase II (Roche) and DNase I (Sigma) for 10 min at 37° C., under agitation. The reaction is stop by adding fresh medium with 7% serum. To generate a single cell suspension, digested tumor tissue was subsequently filtered through 40-μm cell strainers to remove large clumps and debris. This step was repeated using a 15-μm cell strainer. Within a tumor, malignant cells usually recruit other cell types such as immune cells, fibroblasts and endothelial cells to form a complex ‘ecosystem’ that fosters their progression. This ‘ecosystem’ may alter the biological interpretation of our results. However, preliminary determination of immune cell infiltrate by inspection of CD45 revealed that CD45+ positive cells were <10% in all human primary uveal melanomas analyzed indicating that uveal melanomas don't have much stromal tissue. Thus, to avoid cellular damage and delay in cell analysis associated with FACS-sorting, non-melanoma infiltrate was not removed from the cell suspension. This was also supported by data from scRNA-seq showing that primary uveal melanomas display a high purity level as unsupervised transcriptional analysis identified only 101 (<2%) non melanoma cells (13 for patient 1, 4 for patient 2, 11 for patient 3, 14 for patient 4, 8 for patient 5 and 51 for patient 6) with increased expression of immune, fibroblasts or endothelial genes.

Pelleted cells were then resuspended in PBS, counted with the Scepter™ cell counter (Merck Millipore). Cell density was adjusted to 300 cells/μl. Dissociated single cells were stained for viability using the LIVE/DEAD© Viability/Cytotoxicity Kit for mammalian cells (Life Technologies).

Library Preparation and Sequencing.

We followed the 10λ Genomics's protocol (Chromium Single Cell 3′ Reagent Kit, v2 Chemistry) to obtain single cell 3′ libraries for Illumina sequencing. Libraries were sequenced with a NextSeq 500/550 Mid Output v2 kit (150 cycles) using the following read length: 26 bases for read 1 that included the cell barcode and the UMI; 98 bases for read 2 that contained the cDNA insert; 8 bases for index reads.

Sequence Data Preprocessing and Computational Analysis.

Single-cell RNA-seq data was analyzed using CellRanger Single-Cell Suite (v2.0.0) to perform sample demultiplexing, barcode processing, mapping to the human genome (build hg38) and single-cell counting with default parameters. Samples were then merged after read depth correction using CellRanger aggregate function with default parameters. In total, 8,291 cells were remained after the aggregation.

Further analysis was performed in R using the Seurat package45. The combined dataset was filtered to exclude cells expressing less than 1,000 genes and cells with more than 30% of mitochondrial UMI counts. Additionally, genes detected in fewer than 3 cells were removed from the analysis. Data were then scale normalized and log-transformed. From the remaining 7,890 cells, the most variable genes were selected based on their expression and dispersion (expression between 0.0125 and 3 and a dispersion greater than 0.5). To reduce dimensionality, principal component analysis (PCA) was applied to the 1,037 variable genes. The first 10 principal components, which explained most of the variation in the dataset, were used to cluster the cells using the graph-based approach FindClusters function in Seurat, with a resolution parameter of 0.5. Graphical representation of cell clusters was achieved using the t-distributed stochastic neighbor embedding algorithm (t-SNE46) for reduction to two dimensions.

Copy Number Variations from Microarray CGH

Copy number variations were determined from whole genomic DNA by microarray CGH (a-CGH). Genomic DNA was extracted from fresh tissue (case LH16.3814) using the Maxwell 16 LEV BLOOD DNA Purification kit (Promega, Madison, WI) or from FFPE tissue (cases (LH17.364, LH17.530, LH17.3554, LH17.3222, LH18.277) using the Maxwell 16 FFPE Plus LEV DNA Purification kit (Promega). The human reference DNA was from Promega. DNA (500 ng) was labeled using the Genomic DNA ULS Labeling Kit (Agilent) and hybridized onto a Sureprint G3 Human CGH microarray 4×180 K, according to the manufacturer's instructions (Agilent). The microarray slide was scanned using a SureScan scanner (Agilent). Images were analyzed using Cytogenomics software v2.9.2.4 (Agilent). We used the reference assembly Consortium Human 37 [GRCh37] (hg19) from February 2009 [Genome Reference Consortium Human 37 [GRCh37]: human genome 19 (hg19), available at http://genome.ucsc.edu/(Accessed [1] on December, 2018). Genomic gain was defined by a log 2 ratio Cy5/Cy3 >0.2 and genomic loss by a log 2 ratio<0.2. The staging inferred from the cytogenetic data was based on the study of Trolet et al. 47.

Copy Number Inference from Single Cell RNA-Seq Data

To identify the chromosomal variations of each individual cell, we inferred the copy number variations for each cell from the single-cell RNA-seq data using the InferCNV package 15. Briefly, raw UMI counts were transformed in counts-per-10 k and CNVs were estimated by sorting genes by their chromosomal location and centering data by removing the average expression of the reference. The pool of all putative Non-malignant cells from the six patients, supposed to have no chromosomal alterations, were used as the reference for InferCNV. The chromosomal expression patterns were computed by applying moving averages with a sliding window of 150 genes within each chromosome.

SCENIC Analysis

The analysis of regulon activity was performed using SCENIC 16 following the standard pipeline (SCENIC version 0.1.5 which corresponds to RcisTarget 0.99.0, GENIE3 0.99.3 and AUCell 0.99.1; with RcisTarget.hg19.motifDatabases.20 k). The raw gene expression matrix was used as input for SCENIC, from which 9,115 genes passed the default filtering (sum of expression >3*0.01*8,291 and detected in at least 1% of the cells). The co-expression of transcription factors and their putative target genes obtained by GENIE3 was then analyzed for motif enrichment with RcisTarget to build regulons. Only motifs with a normalized enrichment score (NES)>3 were considered as significantly enriched. The regulon activity was analyzed by AUCell and thresholds were modified to the 75^{th} percentile of the normal distribution.

TCGA Analysis

We used the Uveal Melanoma (UM) dataset from The Cancer Genome Atlas (TCGA). Bulk RNA-seq and clinical data were available for 80 patients and downloaded from the TCGA data portal (https://portal.gdc.cancer.gov). RNA-seq data were normalized using the Bioconductor package DESeq2 and log 2 transformed. We also downloaded copy number alterations data from cBioPortal (www.cbioportal.org) to draw the heatmap. EasyROC (http://www.biosoft.hacettepe.edu.tr/easyROC/) was used to plot the ROC and determine the Youden index.

Whole Exome Sequencing

Genomic DNA was prepared from patient's blood sample using the DNeasy Blood and Tissue Kit (Qiagen ref #69504). Whole-exome capture and high-throughput sequencing (HTS) were performed by the Novogene Bioinformatics Institute (Beijing, China). Briefly, a total amount of 1.0 g genomic DNA per sample was used as input material for the DNA library preparation. Libraries were generated using Agilent SureSelect Human All Exon V6 kit (Agilent Technologies, CA, USA) following manufacturer's recommendations and whole exomes were sequenced on the Illumina HiSeq 4000 platform. The sequenced reads were aligned to the human reference genome (UCSC hg19) using Burrows-Wheeler Aligner (BWA) software48. Aligned reads were realigned to the genome. Briefly, duplicates were marked using MarkDuplicates from Picard tools. Indelrealigner and RealignerTargetCreator functions from Genome Analysis Toolkit (GATK) were used to do realignment around the indels according to GATK best practice 49. To avoid system bias, base quality score recalibration was performed with GATK. After realignment to genome, variants (SNPs) were found and filtered using GATK HaplotypeCaller and variantFiltration. Variants obtained from previous steps were annotated with ANNOVAR⁵⁰.

Cell Cultures and Reagents

Human uveal melanoma cell lines Mel27051, 92.152, OMM2.551 were grown in RPMI glutamax supplemented with 10% FBS, OMM153 were grown in DMEM glutamax supplemented with Sodium Pyruvate 1%, MEM Essential Vitamin Mixture 1%, NEAA 1%, Hepes 1%, 10% FBS, MP46, MP65 (from ATCC) were grown in DMEM/F12 w/Glutamine 10% FBS, Glutamax 2 mM, Insulin-Transferin-Selenium 0.5× at 37° C. in a humidified atmosphere containing 5% CO2. Lipofectamine™ RNAiMAX and opti-MEM medium were purchased from Invitrogen (San Diego, CA, USA). BMS-906024 (#BM0018) and DAPT (#5942) were obtained from Sigma, DLL4 from R&D systems (#1506-D4-050).

mRNA Preparation and Real-Time/Quantitative PCR

The mRNAs were prepared using TRIzol (Fisher Scientific, 15596026T) according to a standard procedure. QRT-PCR was performed using SYBR® Green I (Fisher Scientific, 4368708) and Multiscribe Reverse Transcriptase (Applied Biosystems) and subsequently monitored using the ABI Prism 7900 Sequence Detection System (Applied Biosystems, Foster City, CA). The detection of the hRSP14 gene was used to normalize the results. Primer sequences for each cDNA were designed using either Primer bank (https://pga.mgh.harvard.edu/primerbank/). Sequences are available upon request.

Colony Formation Assay

Human melanoma cells were seeded onto 6-well plates and proceed as previously reported 54. The cells were transfected with the siRNA and next placed in a 37° C., 5% CO2 incubator for 7 days. Then, the colonies were stained with 0.04% crystal violet/2% ethanol in PBS for 30 min. Photographs of the stained colonies were captured. Crystal violet was then dissolved and growth was monitored by measuring the absorbance at 405 nm. The colony formation assay was performed in triplicate.

3D Cell Culture

Primary uveal melanoma cells were seeded at a density of 5,000 cells per well and grown for Mel270 cells in DMEM/F12 medium (Thermofisher Scientific, #31331028) supplemented with 20 ng/ml EGF (Peprotech, AF-100-15-500) and 20 ng/ml FGF2 (Peprotech, 100-18C) and for 92.1 cells in MEF medium (R&D systems, #AR005) supplemented with 4 ng/ml FGF2 in low adherence plates. The following day, wells were supplemented with doxycycline 1 μg/ml.

Boyden Chamber Experiments

Cell migration was assessed using a modified Boyden chamber assay with 8-μm pore filter inserts for 24-well plates (BD Bioscience). Cells were seeded on the upper chamber of a trans-well and DMEM 7% FBS placed into the lower chamber. Cells adherent to the underside of the filters were fixed with 4% PFA, stained with 0.4% crystal violet and five random fields at ×20 magnification were counted. Results represent the average of triplicate samples from three independent experiments.

CAM Assays

Chicken egg CAM assays were performed by Inovotion (Grenoble, France). Briefly, fertilized White Leghorn eggs will be incubated at 37.5° C. with 50% relative humidity for 9 days. On day E9, 1×106 92.1 uveal melanoma cells were detached with trypsin, washed with complete medium, suspended and were added onto the CAM of each egg. 30 eggs were used for each group. Embryonic viability was checked daily with an ovoscope. Because some embryo deaths may occur after tumor grafting or may be related to a defective tumor graft, data may be collected with less than 20 eggs per group (minimum of 15 eggs per group). On day E18, the upper portion of the CAM (with tumor) was removed, washed by PBS buffer and then directly transferred in PFA for 48 h (n=15). Next, tumors were carefully cut away from normal CAM tissue and weighed. On day E18, a 1 cm2 portion of the lower CAM was collected to evaluate the number of metastatic cells (n=8). Genomic DNA is extracted from the lower CAM (commercial kit) and analyzed by qPCR with specific primers for Human Alu sequences.

Statistical Analyses

The following statistical tests were used to analyse the data: Pearson correlation, principal component analysis, and paired t-test. When appropriated, statistical difference between groups is made visible on graphs by the presence of stars with the following meaning:
No star: No statistical difference (p-value>0.05);
*: 0.05≥p-value>0.01;
**: 0.01≥p-value>0.001;
***: 0.001≥p-value.

Results

To inspect intratumoral heterogeneity, we isolated individual cells from six freshly resected human primary uveal melanomas and generated single cell transcriptomes using 10× genomics (data not shown). The basal diameter of the tumors averaged 16.0 mm (range, 10.0-19.0) and no tumors displayed extrascleral extension (data not shown). Histological examination showed epithelioid cells in tumors 2 and 3, and a high mitotic index in tumor 2 (data not shown). Whole exome sequencing of bulk lesions revealed that they displayed a classical mutational landscape as expected from previous data 8, i.e mutually exclusive mutations involving GNAQ and GNA11 in all the cases and alterations affecting SF3B1 and BAP1 that tended to not overlap. Histopathological and cytogenetic features that are used to classify the lesions and to provide a metastatic risk are shown for the 6 primary uveal melanomas (data not shown).

We first used the principal component analysis (PCA) and examined the two first principal components, which we observed constituted the majority of the variance within the dataset (data not shown). Among the 10 genes most strongly associated with PC1 was HTR2B, a previously reported poor prognosis gene⁹.

Cellular function or disease analysis using Ingenuity® Pathway Analysis (IPA) software indicated that the PC1 signature was related to cell movement of tumor cell lines, migration of tumor cell lines, cell viability, cell survival, neoplasia of cells. Instead cellular functions or diseases related to apoptosis or necrosis were inhibited (data not shown). Interestingly, liver tumor function was also predicted and might be in line with the liver tropism commonly observed for primary uveal melanomas. IPA revealed that PC2 was linked to proliferation of tumor cells and invasion of tumor (data not shown).

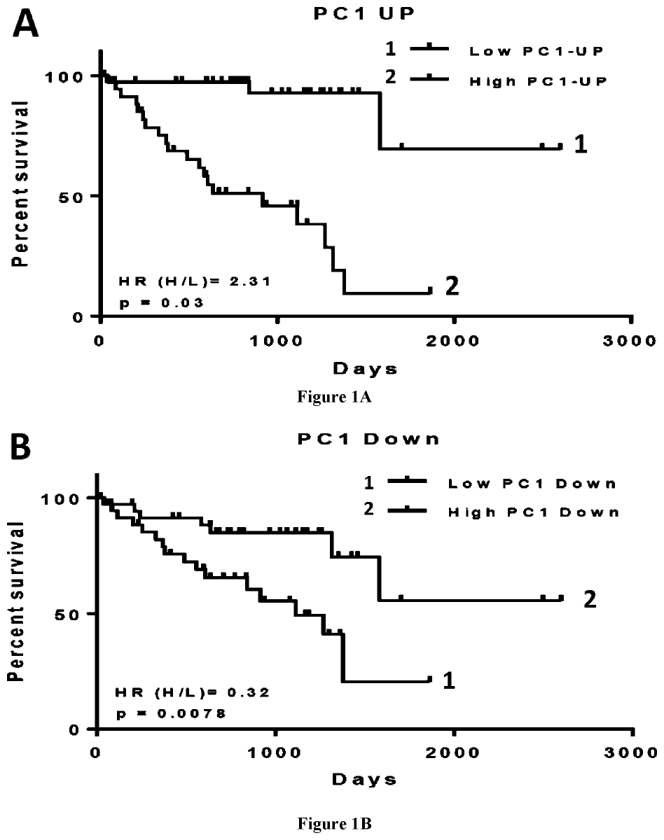

Kaplan-Meier analysis of uveal melanoma patients (TCGA set) showed that expression of the top 10 genes with the highest PC1 scores was associated with shortened survival (FIG. 1A), whereas expression of the top 10 genes with the lowest PC1 scores correlated with a long-term survival (FIG. 1). PC1 also displayed RAB31, CDH1, PTP4A3 and PRAME (data not shown), which were previously associated with poor prognosis in primary uveal melanomas⁹⁻¹¹. Although to a lesser extent, expression of the top 10 genes with the highest scores in PC2 was also predictive of a poor prognosis (data not shown).

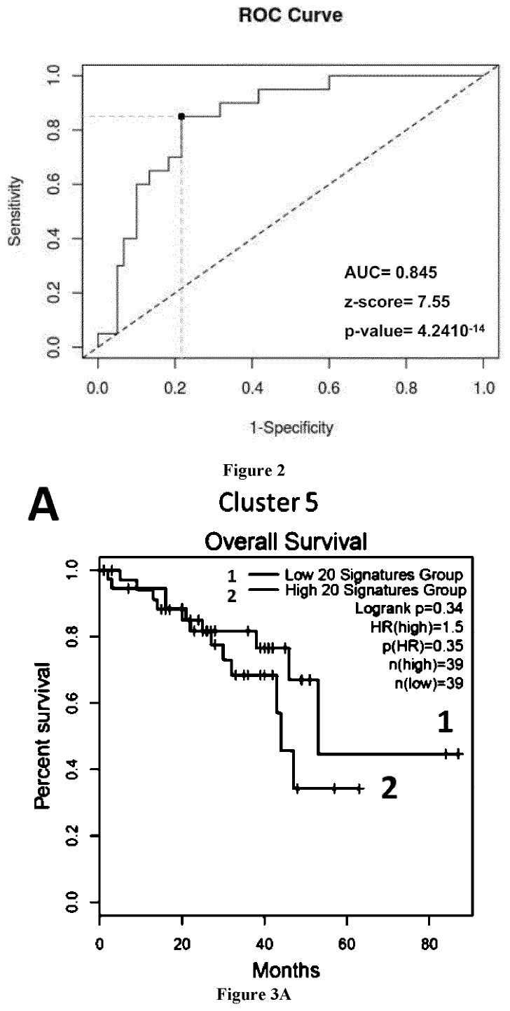

To estimate the prognosis ability of PC1 genes, we used the top 10 up and down genes to calculate a PC1 score for patients in the TCGA cohort. Next, we plotted a ROC curve and evaluated the Youden index (FIG. 2). The AUROC was 0.84 and the Youden index 0.63, thereby indicating that this PC1 score might be of interest to estimate patients' prognosis. If we extrapolate this to the single cell analysis, cells with a PC1 score above the Youden index should be endowed with aggressive tumorigenic properties and convey a “poor prognosis”, while those with a PC1 score under the Youden index should be associated “good prognosis”.

Applying this concept, we found that tumors 1, 3 and 5 from patients at metastatic risk (subgroup 2c) contained between 80% to 100% of “poor prognosis cells”, while tumor 2 (subgroup 2a) contained only 20% (data not shown). Among tumors with favorable outcome, tumors 4 and 6 comprised only 0.8 and 3.5% of poor prognosis cells, respectively. However, this small number of cells might be sufficient to support distant metastasis development and impact on the patient's outcome (data not shown).

To identify salient biological cell states, we next performed clustering of the individual cells with the Seurat analysis pipeline and used non-linear dimensionality reduction method [t-distributed stochastic neighbor embedding (t-SNE)], to visualize the cell clusters. This analysis revealed that most cells grouped by tumor of origin, thereby indicating inter-tumor heterogeneity (data not shown).

Further unbiased clustering of the individual cells identified 11 clusters (data not shown). Tumors 2, 4 and 5 each comprised a single cluster (data not shown), while 2 clusters were identified in tumors 1 and 6, and 3 clusters in tumor 3, suggesting various levels of intra-tumor heterogeneity. Few non-malignant cells were detected in the tumors. Cluster 11 was annotated as immune cells since it was enriched in the expression of T cells and monocytes/macrophages markers and cluster 9 as endothelial cells since it was enriched in the expression of PECAM1, CD34, FLT1, CDH5 (data not shown). These two latter clusters gathered by cell type and not by patient. Thus, our data indicate that the primary uveal melanomas analysed in this study are composed of multiple cell types. They don't show much stromal tissue, in agreement with what has been previously reported¹².

List of genes associated with each cluster (data not shown) and was used in IPA comparison analysis to address enrichment in canonical pathways. Clusters 2, 4, 7, 8 and 10 clustered together and disclosed clear activation of Rho GTPase-dependent signaling pathways, regulation of actin cytoskeleton and integrin signaling (data not shown). Consistently, in these clusters, Rho-GDI, a negative regulator of signaling through Rho GTPases was down-regulated. Rho GTPases are essential in propagating integrin-mediated responses and, by tightly regulating actin cytoskeleton, offer a key signaling link through which adhesion, spreading, and migration are controlled in tumor cells¹³. Other pathway more robustly expressed in these clusters included mitochondria oxidative phosphorylation (data not shown), which is also linked to cancer cell migration¹⁴.

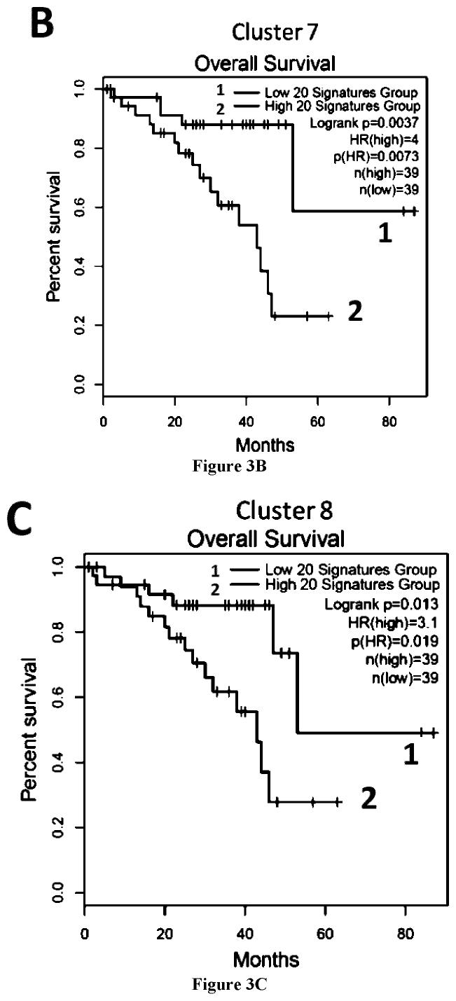

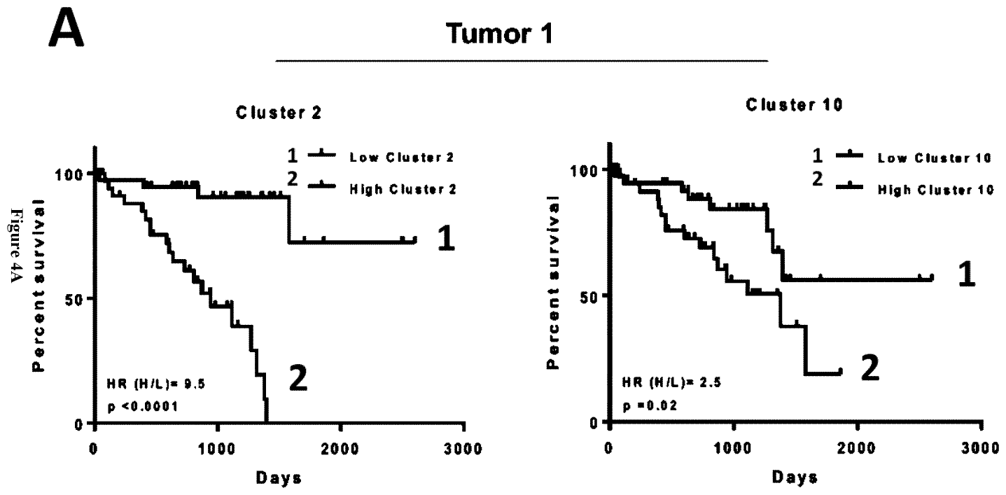

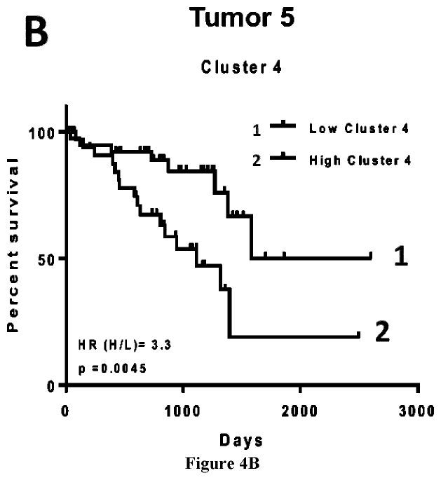

In keeping with the recognized role of Rho GTPases and mitochondrial metabolism as markers of tumor invasion and metastasis, Kaplan-Meier survival plot generated from the top 25 genes in each cluster revealed that only clusters 2, 4, 7, 8 and 10 were associated with a poor prognosis (FIG. 3A-C) and FIG. 4A-B). Thus, in tumor 3, whereas cluster 5 was not related to the prognosis, clusters 7 and 8 contained cells conveying a dismal prognosis (FIG. 3A-C), further supporting the existence of transcriptomic and functional heterogeneity in primary uveal melanomas.

In addition, as previously described¹⁵, large-scale copy number aberrations for each cell by averaging relative expression levels over large genomic regions was used to infer copy number variation (CNV) from scRNA-seq data (data not shown). Inferred-CNV profiles identified distinct chromosomal imbalance, including chromosome 3 loss and 8q gain, that are characteristic of uveal melanomas. Globally, inferred-CNV analysis was in agreement with bulk array-based comparative genomic hybridization (a-CGH) (data not shown). However, tumors 3 and 6 appeared to contain more than one genetic clone, as visualized by copy number changes on several chromosomic regions in a subset of cells from the same patient. Cryptic alterations, in cell subsets of patients 1 and 4 can be observed in chromosomes 6 and 8 respectively.

Collectively, the single cell data analysis revealed, in addition to inter-tumor heterogeneity, a high degree of intra-tumoral heterogeneity at both the genetic and transcriptomic level. Transcriptomic and genetic heterogeneity overlapped largely in tumor 6 where the cells with 8q gain fell in cluster 6, and in tumor 3 where cells having a loss in chromosomes 14, 15 and 16 segregated to cluster 7. However, in tumor 3, cells with a 6p gain were distributed between cluster 8 and a portion of cluster 5. The intra-tumor heterogeneity highlighted in these single-cell analyses could therefore represent a source of inaccuracy in uveal melanoma staging, and patient prognosis.

Next, to get insights into the stable transcriptional cell states, we used the Single-cell regulatory network inference and clustering (SCENIC) method¹⁶. SCENIC exploits transcription factors and cis-regulatory sequences, to map the activity of the regulatory networks (regulons) underlying the different gene expression signatures. This analysis disclosed 122 regulons (out of 1046) that displayed significant activity (data not shown). In contrast to standard clustering, SCENIC showed degree of cellular overlapping between cells from different tumors (data not shown) and identified three main uveal melanoma transcriptional states (data not shown).

SCENIC predicted a cell specification and differentiation state driven by SOX (SOX4/5/6/9/10) and ETS transcription factors (ETS1, ETV5, ELK4, GABPA). This state overlapped with cells harboring activity of MITF a master regulator of melanocyte development, function and survival^{17, 18}. Accordingly, SOX10, as well as PAX3 which regulon was also enriched in this cell state, are major drivers of MITF. MITF expression has been associated with melanocytic cell differentiation¹⁹. This state correlated with a low PC1 score and mainly tagged tumor 4 (data not shown).

Finally, a proliferative state was inferred from enrichment in immediate-early genes (JUNB, JUND, FOS, FOSB). FOS, a member of the AP-1 complex, was recently shown to be a direct YAP/TAZ transcriptional target²⁶. YAP/TAZ activation drives uveal melanoma progression 27. The proliferative state was enriched in tumors 1, 2, 3 and was associated with a moderate to high PC1 score. MITF activity was detected in a large proportion of cells from tumors 1 and 2 in agreement with its role in regulating proliferation of cutaneous melanoma²⁸. Instead, a fraction of tumor 3 cells became completely devoid of any MITF activity. JUN transcription factors are important drivers of the MITF-low state²⁹, which in cutaneous melanoma, was associated with motile ability and resistance to treatment³⁰⁻³². Note that a cell subpopulation of tumors 1 and 3 in the proliferative state overlapped with the invasive state. This cell subpopulation also displayed low levels of SOX10 transcriptional activity, which correlated with stem-like properties in cutaneous melanoma cells³³ and may thus play a prominent role in driving metastasis. Note that MITF can display an ambivalent role, since depending on the context, MITF can also stimulate invasion^{34, 35} and both high and low MITF was associated with drug resistance^{36, 37}.

Thus, these analyses indicated that the transcriptional states are not uniform from one patient's tumor to another, that all showed a mix of variable proportions and distinct cell subpopulations and demonstrated intra-tumor heterogeneity.

### REFERENCES

Throughout this application, various references describe the state of the art to which this invention pertains. The disclosures of these references are hereby incorporated by reference into the present disclosure.
1. Nabil A A, Marie S, Marc-Henri S, et al: Upcoming translational challenges for uveal melanoma [Internet]. Br J Cancer 113:1249-1253, 2015 Available from: http://dx.doi.org/10.1038/bjc.2015.269
2. Pandiani C, Beranger G E, Leclerc J, et al: Focus on cutaneous and uveal melanoma specificities [Internet]. Genes Dev 31:724-743, 2017 Available from: http://www.ncbi.nlm.nih.gov/pubmed/28512236
3. Yang J, Manson D K, Marr B P, et al: Treatment of uveal melanoma: where are we now? Ther Adv Med Oncol, 2018
4. Rietschel P, Panageas K S, Hanlon C, et al: Variates of survival in metastatic uveal melanoma [Internet]. J Clin Oncol 23:8076-8080, 2005 Available from: http://www.ncbi.nlm.nih.gov/pubmed/16258106
5. Tirosh I, Izar B, Prakadan S M, et al: Dissecting the multicellular ecosystem of metastatic melanoma by single-cell RNA-seq [Internet]. Science (80-) 352:189-196, 2016 Available from: http://www.ncbi.nlm.nih.gov/pubmed/27124452
6. Kemper K, Krijgsman O, Cornelissen-Steijger P, et al: Intra- and inter-tumor heterogeneity in a vemurafenib-resistant melanoma patient and derived xenografts [Internet]. EMBO Mol Med 7:1104-1118, 2015 Available from: http://www.ncbi.nlm.nih.gov/pubmed/26105199
7. Kemper K, de Goeje P L, Peeper D S, et al: Phenotype switching: tumor cell plasticity as a resistance mechanism and target for therapy [Internet]. Cancer Res 74:5937-5941, 2014 Available from: http://www.ncbi.nlm.nih.gov/pubmed/25320006
8. Shain A H, Bagger M M, Yu R, et al: The genetic evolution of metastatic uveal melanoma [Internet]. Nat Genet, 2019 Available from: http://www.nature.com/articles/s41588-019-0440-9
9. Onken M D, Worley L A, Ehlers J P, et al: Gene expression profiling in uveal melanoma reveals two molecular classes and predicts metastatic death [Internet]. Cancer Res 64:7205-7209, 2004 Available from: http://www.ncbi.nlm.nih.gov/pubmed/15492234
10. Laurent C, Valet F, Planque N, et al: High PTP4A3 phosphatase expression correlates with metastatic risk in uveal melanoma patients [Internet]. Cancer Res 71:666-674, 2011 Available from: https://www.ncbi.nlm.nih.gov/pubmed/21135111
11. Field M G, Decatur C L, Kurtenbach S, et al: PRAME as an Independent Biomarker for Metastasis in Uveal Melanoma [Internet]. Clin Cancer Res 22:1234-1242, 2016 Available from: http://www.ncbi.nlm.nih.gov/pubmed/26933176
12. Bronkhorst I H G, Jager M J: Uveal melanoma: The inflammatory microenvironment. J Innate Immun 4:454-462, 2012
13. Lawson C D, Burridge K: The on-off relationship of Rho and Rac during integrin-mediated adhesion and cell migration. Small GTPases 5, 2014
14. Zhao J, Zhang J, Yu M, et al: Mitochondrial dynamics regulates migration and invasion of breast cancer cells. Oncogene 32:4814-4824, 2013
15. Patel A P, Tirosh I, Trombetta J J, et al: Single-cell RNA-seq highlights intratumoral heterogeneity in primary glioblastoma [Internet]. Science (80-) 344:1396-1401, 2014 Available from: https://www.ncbi.nlm.nih.gov/pubmed/24925914
16. Aibar S, Gonzalez-Blas C B, Moerman T, et al: SCENIC: single-cell regulatory network inference and clustering [Internet]. Nat Methods 14:1083-1086, 2017 Available from: http://www.ncbi.nlm.nih.gov/pubmed/28991892
17. Goding C R, Arnheiter H: MITF the first 25 years 983-1007, 2019
18. Cheli Y, Ohanna M, Ballotti R, et al: Fifteen-year quest for microphthalmia-associated transcription factor target genes. Pigment Cell Melanoma Res 23, 2010
19. Rambow F, Marine J, Goding C R: Melanoma plasticity and phenotypic diversity: therapeutic barriers and opportunities 1295-1318, 2019
20. Meir T, Dror R, Yu X, et al: Molecular characteristics of liver metastases from uveal melanoma [Internet]. Invest Ophthalmol Vis Sci 48:4890-4896, 2007 Available from: http://www.ncbi.nlm.nih.gov/pubmed/17962435
21. Singh M, Kashyap S, Singh L, et al: Loss of BAP-1 influences the activation of p52 and RelB proteins in the Inflammatory microenvironment of uveal melanoma. Ann Oncol 30 (suppl_:xi48-xi57, 2019
22. Gao K, Dai D L, Martinka M, et al: Prognostic significance of nuclear factor-κB p105/p50 in human melanoma and its role in cell migration. Cancer Res 66:8382-8388, 2006
23. Xu Y, Liu X, Zhang H, et al: Overexpression of HES6 has prognostic value and promotes metastasis via the Wnt/beta-catenin signaling pathway in colorectal cancer [Internet]. Oncol Rep 40:1261-1274, 2018 Available from: https://www.ncbi.nlm.nih.gov/pubmed/30015909
24. Haapa-Paananen S, Kiviluoto S, Waltari M, et al: HES6 gene is selectively overexpressed in glioma and represents an important transcriptional regulator of glioma proliferation [Internet]. Oncogene 31:1299-1310, 2012 Available from:
   https://www.ncbi.nlm.nih.gov/pubmed/21785461
25. Wickramasinghe C M, Domaschenz R, Amagase Y, et al: HES6 enhances the motility of alveolar rhabdomyosarcoma cells [Internet]. Exp Cell Res 319:103-112, 2013 Available from: https://www.ncbi.nlm.nih.gov/pubmed/22982728
26. Koo J H, Plouffe S W, Meng Z, et al: Induction of AP-1 by YAP/TAZ contributes to cell proliferation and organ growthl-15, 2020
27. Li H, Li Q, Dang K, et al: YAP/TAZ Activation Drives Uveal Melanoma Initiation and Progression. Cell Rep 29:3200-3211.e4, 2019
28. Du J, Widlund H R, Horstmann M A, et al: Critical role of CDK2 for melanoma growth linked to its melanocyte-specific transcriptional regulation by MITF [Internet]. Cancer Cell 6:565-576, 2004 Available from: http://www.ncbi.nlm.nih.gov/entrez/query.fcgi?cmd=Retrieve&db=PubMed&dopt=Citation&list_uids=15607961
29. Verfaillie A, Imrichova H, Atak Z K, et al: Decoding the regulatory landscape of melanoma reveals TEADS as regulators of the invasive cell state [Internet]. Nat Commun 6:6683, 2015 Available from: http://www.ncbi.nlm.nih.gov/pubmed/25865119
30. Garraway L A, Weir B A, Zhao X, et al: “Lineage addiction” in human cancer: lessons from integrated genomics [Internet]. Cold Spring Harb Symp Quant Biol 70:25-34, 2005 Available from: http://www.ncbi.nlm.nih.gov/pubmed/16869735
31. Akavia U D, Litvin O, Kim J, et al: An integrated approach to uncover drivers of cancer [Internet]. Cell 143:1005-1017, 2010 Available from: http://www.ncbi.nlm.nih.gov/pubmed/21129771
32. Muller J, Krijgsman O, Tsoi J, et al: Low MITF/AXL ratio predicts early resistance to multiple targeted drugs in melanoma [Internet]. Nat Commun 5:5712, 2014 Available from: http://www.ncbi.nlm.nih.gov/pubmed/25502142
33. Sun C, Wang L, Huang S, et al: Reversible and adaptive resistance to BRAF(V600E) inhibition in melanoma [Internet]. Nature 508:118-122, 2014 Available from: http://www.ncbi.nlm.nih.gov/pubmed/24670642
34. McGill G G, Haq R, Nishimura E K, et al: c-Met expression is regulated by Mitf in the melanocyte lineage. J Biol Chem 281:10365-10373, 2006
35. Bertolotto C, Lesueur F, Giuliano S, et al: A SUMOylation-defective MITF germline mutation predisposes to melanoma and renal carcinoma. Nature 480, 2011
36. Ngeow K C, Friedrichsen H J, Li L, et al: BRAF/MAPK and GSK3 signaling converges to control MITF nuclear export. Proc Natl Acad Sci USA 115:E8668-E8677, 2018
37. Johannessen C M, Johnson L A, Piccioni F, et al: A melanocyte lineage program confers resistance to MAP kinase pathway inhibition [Internet]. Nature 504:138-142, 2013 Available from: http://www.ncbi.nlm.nih.gov/pubmed/24185007
38. Gaiano N, Fishell G: The role of notch in promoting glial and neural stem cell fates [Internet]. Annu Rev Neurosci 25:471-490, 2002 Available from: https://www.ncbi.nlm.nih.gov/pubmed/12052917
39. Kopan R, Ilagan M X G: The Canonical Notch Signaling Pathway: Unfolding the Activation Mechanism. Cell 137:216-233, 2009
40. Asnaghi L, Ebrahimi K B, Schreck K C, et al: Notch signaling promotes growth and invasion in uveal melanoma. Clin Cancer Res 18:654-665, 2012
41. Asnaghi L, Handa J T, Merbs S L, et al: A role for Jag2 in promoting uveal melanoma dissemination and growth. Investig Ophthalmol Vis Sci 54:295-306, 2013
42. Visvader J E, Lindeman G J: Cancer stem cells: Current status and evolving complexities [Internet]. Cell Stem Cell 10:717-728, 2012 Available from: http://dx.doi.org/10.1016/j.stem.2012.05.007
43. Dopierala J, Damato B E, Lake S L, et al: Genetic heterogeneity in uveal melanoma assessed by multiplex ligation-dependent probe amplification [Internet]. Invest Ophthalmol Vis Sci 51:4898-4905, 2010 Available from: http://www.ncbi.nlm.nih.gov/pubmed/20484589
44. Roma J, Masia A, Reventós J, et al: Notch pathway inhibition significantly reduces rhabdomyosarcoma invasiveness and mobility in vitro. Clin Cancer Res 17:505-513, 2011
45. Satija R, Farrell J A, Gennert D, et al: Spatial reconstruction of single-cell gene expression data [Internet]. Nat Biotechnol 33:495-502, 2015 Available from: http://www.ncbi.nlm.nih.gov/pubmed/25867923
46. Van Der Maaten L, Hinton G: Visualizing data using t-SNE. J Mach Learn Res, 2008
47. Trolet J, Hupe P, Huon I, et al: Genomic profiling and identification of high-risk uveal melanoma by array CGH analysis of primary tumors and liver metastases [Internet]. Invest Ophthalmol Vis Sci 50:2572-2580, 2009 Available from: http://www.ncbi.nlm.nih.gov/pubmed/19151381
48. Li H, Durbin R: Fast and accurate short read alignment with Burrows-Wheeler transform [Internet]. Bioinformatics 25:1754-1760, 2009 Available from: http://www.ncbi.nlm.nih.gov/pubmed/19451168
49. Van der Auwera G A, Carneiro M O, Hartl C, et al: From fastQ data to high-confidence variant calls: The genome analysis toolkit best practices pipeline. Curr Protoc Bioinforma, 2013
50. Wang K, Li M, Hakonarson H: ANNOVAR: Functional annotation of genetic variants from high-throughput sequencing data. Nucleic Acids Res 38:1-7, 2010
51. Chen P W, Murray T G, Uno T, Salgaller M L, Reddy R K B: Expression of MAGE genes in ocular melanoma during progression from primary to metastatic disease. Clin Exp Metastasis 15:509-18, 1997
52. De Waard-Siebinga I, Blom D R, Griffioen M, et al: Establishment and characterization of an uveal-melanoma cell line. Int J Cancer, 1995
53. Luyten G P, Naus N C, Mooy C M, et al: Establishment and characterization of primary and metastatic uveal melanoma cell lines [Internet]. Int J Cancer 66:380-387, 1996 Available from: https://www.ncbi.nlm.nih.gov/pubmed/8621261
54. Ohanna M, Cerezo M, Nottet N, et al: Pivotal role of NAMPT in the switch of melanoma cells toward an invasive and drug-resistant phenotype [Internet]. Genes Dev 32:448-461, 2018 Available from: https://www.ncbi.nlm.nih.gov/pubmed/29567766

1. A method for predicting the survival time of a subject suffering from uveal melanoma and/or metastatic uveal melanoma and treating the subject, comprising the steps of:
i) determining a score of SPP1, EMCN, SYNPR, CTC-340A15.2, HPGD, MTRNR2L8, PDE4DIP, COX6A2, AHCYL2, GSTA3 and H3F3A in a biological sample obtained from the subject;
ii) administering, to a subject identified as having a score of SPP1, EMCN, SYNPR, CTC-340A15.2, HPGD, MTRNR2L8, PDE4DIP, COX6A2, AHCYL2, GSTA3 and H3F3A that is higher than a corresponding predetermined reference value, a therapeutically effective amount of an activator of SPP1, EMCN, SYNPR, CTC-340A15.2, HPGD, MTRNR2L8, PDE4DIP, COX6A2, AHCYL2, GSTA3 and H3F3A.

2. The method according to claim 1, wherein the score of SPP1, EMCN, SYNPR, CTC-340A15.2, HPGD, MTRNR2L8, PDE4DIP, COX6A2, AHCYL2, GSTA3 and H3F3A is determined by an RNA fluorescence in situ hybridization assay.

3. The method according to claim 1, wherein the biological sample is blood sample or tumor biopsy sample.

4. A method for treating uveal melanoma and/or metastatic uveal melanoma in a subject in need thereof comprising administering to the subject a therapeutically effective amount of an activator of SPP1, EMCN, SYNPR, CTC-340A15.2, HPGD, MTRNR2L8, PDE4DIP, COX6A2, AHCYL2, GSTA3 and/or H3F3A.

5. The method according to claim 4, wherein the subject is identified as having a bad prognosis.

6. The method according to claim 4, wherein the activator of SPP1, EMCN, SYNPR, CTC-340A15.2, HPGD, MTRNR2L8, PDE4DIP, COX6A2, AHCYL2, GSTA3 and/or H3F3A is selected from the group consisting of: a small organic molecule, an aptamer, an antibody, a peptide or a polypeptide.

7. The method according to claim 1, wherein the uveal melanoma is resistant to a treatment with inhibitors of BRAF mutations, inhibitors of MEK, inhibitors of NRAS or inhibitors of an immune checkpoints.

8. The method according to claim 1, wherein the activator of SPP1, EMCN, SYNPR, CTC-340A15.2, HPGD, MTRNR2L8, PDE4DIP, COX6A2, AHCYL2, GSTA3 and/or H3F3A is administered in combination with radiation therapy, immunotherapy or chemotherapy.

9. The method according to claim 1, wherein the activator of SPP1, EMCN, SYNPR, CTC-340A15.2, HPGD, MTRNR2L8, PDE4DIP, COX6A2, AHCYL2, GSTA3 and/or H3F3A is administered in combination with an immune checkpoint inhibitor.

10. The method according to claim 1, wherein the activator of SPP1, EMCN, SYNPR, CTC-340A15.2, HPGD, MTRNR2L8, PDE4DIP, COX6A2, AHCYL2, GSTA3 and/or H3F3A is administered in combination with a BRAF inhibitor.

11. The method according to claim 1, wherein the activator of SPP1, EMCN, SYNPR, CTC-340A15.2, HPGD, MTRNR2L8, PDE4DIP, COX6A2, AHCYL2, GSTA3 and/or H3F3A is administered in combination with a MEK inhibitor.

12. A kit for use in the method according to claim 1, said kit comprising i) a reagent that specifically reacts with SPP1, EMCN, SYNPR, CTC-340A15.2, HPGD, MTRNR2L8, PDE4DIP, COX6A2, AHCYL2, GSTA3, H3F3A mRNA or protein and ii) instructions for use in treating uveal melanoma and/or metastatic uveal melanoma.

13. A composition comprising i) an inhibitor of SPP1, EMCN, SYNPR, CTC-340A15.2, HPGD, MTRNR2L8, PDE4DIP, COX6A2, AHCYL2, GSTA3 and/or H3F3A, and ii) an immune checkpoint inhibitor, a BRAF inhibitor and/or a MEK inhibitor.
